(19) United States
(12) Patent Application Publication
(10) Pub. No.: US 2016/0361264 A1
(43) Pub. Date: Dec. 15, 2016

(54) DELIVERY OF PHARMACEUTICAL ACTIVE INGREDIENTS THROUGH THE SKIN AND HAIR FOLLICLES INTO DERMIS AND TRANSDERMAL DELIVERY

(71) Applicant: SALVONA LLC, Hamilton, NJ (US)

(72) Inventors: Samuel D. SHEFER, East Brunswick, NJ (US); Harriet L.R. COOPER, Woodbridge, NJ (US); Nicholas POLOGRUTO, New Brunswick, NJ (US)

(21) Appl. No.: 14/738,515

(22) Filed: Jun. 12, 2015

(51) Int. Cl. A61K 9/50; A61K 8/49; A61Q 7/00; A61K 31/19; A61K 31/245; A61K 31/7056; A61K 31/235; A61K 31/60; A61Q 5/00; A61K 31/138; A61K 8/11; A61K 31/327

(52) U.S. Cl. CPC A61K 9/5015; A61K 8/11; A61K 8/4953; A61Q 7/00; A61K 31/19; A61K 31/327; A61K 31/7056; A61K 31/235; A61K 31/60; A61Q 5/006; A61K 31/138; A61K 31/245

(57) ABSTRACT

A targeted nanosphere and submicron controlled delivery system for topical and transdermal use is disclosed. The controlled delivery system can be used as a targeted drug delivery system. The controlled delivery system is a composition of active ingredients encapsulated in sub-micron spheres, optionally with an additional protective shell. The sphere includes a solid core and a semi-liquid shell. The size of the delivery system can be selected to supply the active ingredients in a targeted manner to the skin to treat skin conditions or through follicular penetration in order to treat for example, follicular diseases and stimulate or reduce hair growth.

### BACKGROUND OF THE INVENTION

### 1. Field of the Invention

The current invention relates to the field of targeted drug delivery systems composed of nanospheres and microspheres encapsulated with pharmaceutical or cosmetic active ingredients for topical and transdermal use.

### 2. Description of Related Art

There are two fundamental routes for penetration of active ingredients from topical formulations through the stratum corneum, or horny layers, of the skin: (1) by movement through the intercellular spaces of the keratinocytes or (2) by penetration into the hair follicles. The latter route is especially important for skin diseases that occur within the hair follicles, for instance: acne, folliculitis, hidradentitis suppurativa, and keratosis pilaris. The follicular delivery route is also a route for transdermal delivery, as it allows for faster and deeper delivery than the intercellular route.

WO 2011095970 A1 and US20120301527 A1 disclose a composition to target hair follicles using cosmetic composition with emulsions of an oil-in-polyol with a particle size of below one micron. These compositions have the drawback of containing polyols and are potentially irritating. The oil-in-polyol character of the emulsion limits the nature of actives that can be incorporated, for instance, water-soluble actives would be difficult to incorporate.

WO 2003015729 A1 discloses follicular delivery of an ornithine decarboxylase inhibitor using a follicular delivery-enhancing agent. The enhancing agent comprising for examples, steareth-20, steareth-100, poloxamer 185, poloxamer 407.

WO 2014028608 A1 discloses a zein-shell encapsulation of methotrexate, BPO, and retinoic acid and is used in combination with ethanol, propylene glycol and another organic solvent, such as ethyl acetate. This patent application has the drawback of having potential for irritation.

WO 1994022468 A1 discloses liposome formulations to target hair follicles. Liposomes are not very robust and are not usually a commercially viable option. They generally have poor loading capacity for hydrophobic actives.

There exists a need for targeted delivery systems that treat diseased sites of the skin specifically but do not damage healthy skin, contrasted to treatments not using a delivery system but free actives, where the dosage necessary for efficacy increases the chance of side-effects including but not limited to redness, irritation, and dryness. Furthermore, there exists a need for delivery into the dermis and deeper layers of the epidermis without the use of chemical penetration enhancers such as isopropyl palmitate, propylene glycol, DMSO, and Transcutol. Furthermore, there exists a need for delivery systems that deliver active ingredients through the skin and into the bloodstream, thus bypassing the gastric tract and first-pass metabolism by the liver.

### SUMMARY OF THE INVENTION

The present invention relates to a sub-micron spheroid structure that encapsulates active ingredients and allows slow release in addition to follicular targeting. These structures can be prepared as an aqueous suspension of the sub-micron spheroids or a plurality of spheroids surrounded by a larger outer microsphere in powder form. In addition, the present invention provides stabilizing effects on unstable actives and is readily compatible with formulation into preparations typically employed in topical treatments including but not limited to lotions, creams, unguents, gels, and suspensions.

While conventional active pharmaceutical ingredients that are used topically to treat skin disorders are effective, in many cases cause distressing side effects such as redness, irritation, and sensitivity to UV light can occur due to the large amount of active drug material utilized in the formula to ensure that sufficient amounts are transported across the stratum corneum. This leads to poor patient compliance and insufficient treatment, or adverse events that are as unpleasant or worse than the disease. The present invention enables a reduced dosage that is protected in a delivery system and targets the delivery into the follicles or through the stratum corneum for an accurate site of action.

The drug delivery system of the present invention utilizing submicron solid encapsulation for topical use where the particle size and the composition are optimal for targeting into the hair follicles and that can be used in preparations typically employed in topical treatments including but not limited to lotions, creams, unguents, gels, and suspensions. The composition of the capsule matrix enhances the compatibility of drug material with the lipid environment of the skin.

The drug delivery system comprises a plurality of spheres that may either be suspended in water or enrobed in microsphere shells as a dry powder material. The shell of the submicron sphere comprises a polymer increasing bio-adhesion through electrostatic attraction and hydrophobic interactions to the surfaces of the skin. The delivery system of the present invention provides sustained release properties by the encapsulation of actives into a matrix system that gradually breaks down in the skin to allow the time-release properties. In the embodiment of the invention wherein the submicron solid particles are enrobed and encapsulated within an outer microsphere, the release of the submicron particles can be triggered by external factors including but not limited to hydration, shear and/or frictional forces, changes in pH, or some combination thereof.

The present invention discloses a method to treat skin disorders including but not limited to acne, rosacea, psoriasis, shingles, eczema, atopic and other kinds of dermatitis, fungal and bacterial infections. The present invention provides targeted delivery to the follicles and offers reduced irritation and protection of sensitive active ingredients from oxidation. Furthermore, the present invention provides a delivery system to place an active material at the site of follicular disease without disrupting the membranes of the skin layers via the action of solvents or surfactants, thus reducing the chance of side effects.

The present invention is applicable to a wide range of active ingredients that can be encapsulated in the submicron spheres. The present invention has up to 80% capacity for loading of an active ingredient. The present invention provides a dermatological and trichological drug delivery system for topical use, which is free of volatile organic solvents such as ethanol or methanol. The drug delivery system provides sustained release properties through encapsulation of the actives into a matrix system that gradually breaks down in the skin to allow the time-release properties and allows encapsulation of both hydrophobic and hydrophilic actives.

In addition, it allows for the preparation of non-irritant products by exclusion of chemical penetration enhancers and volatile organic solvents. This decreases the chance of side effects and increases patient compliance, and is thus suitable for sensitive skin

The invention will be more fully described by reference to the following drawings.

### BRIEF DESCRIPTION OF THE DRAWINGS

FIG. 1 is a graph of minoxidil penetration into the skin over a six hour time period as measured in Franz diffusion cells.

FIGS. 2A-2D are graphs of fluorescence microscopy detailing a cross section progression into the skin of the encapsulated material after 24 hours.

FIG. 3 is a graph of a quantitative test assessing penetration depth of invention versus free minoxidil without penetration-enhancing solvents.

FIG. 4 is a graph of decreased penetration of minoxidil due to obstructed follicles.

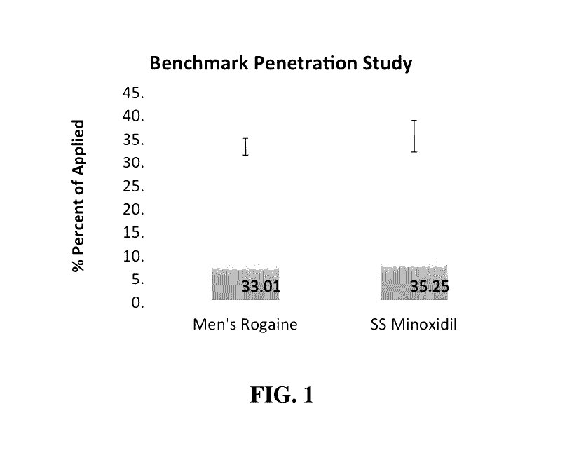

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

The present invention provides a targeted delivery system for topical and transdermal use. The controlled delivery system of the present invention can be used as a drug delivery system. The controlled delivery system of the present invention comprises a composition of active ingredients encapsulated in sub-micron spheres. The term “spheres” is intended to describe spherical particulates. It will be appreciated that other spherical shapes including spheroids can be used in accordance with the teachings of the present invention. Spheres of the present invention can have an average diameter in the range of from about 0.01 micron to about 10 microns. Preferably, the sphere size is in the range from about 0.05 microns to about 5.0 microns. This linear dimension for any individual sphere represents the length of the longest straight line joining two points on the surface of the sphere. The size of the delivery system can be selected to supply the active ingredients in a targeted manner through follicular penetration in order to treat for example, follicular diseases and the acceleration/deceleration of hair growth.

In one embodiment, the sphere comprises a solid core and a semi-liquid shell. The shell can be formed as a coating on the core via physical interactions. In one embodiment, the shell is made of water-suspendable polymers with properties to provide a stable suspension and bioadhesive properties.

In one embodiment, the target delivery system includes between about 0.1% and 100% of one or more lipids; between about 0.001% and 20% of one or more surfactants; between about 0.001% and 80% of one or more active ingredients used to treat one or more disorders; and optionally a solubilizing excipient. The solubilizing excipient can include but is not limited to a non-volatile organic solvent chosen from the group of glycerol esters, alkyl esters, aromatic esters glycerol, glycols, polyalcohols, sorbitol and sorbitol derivatives.

The composition of the core can comprise one or more lipids. Suitable lipids include but are not limited to waxes, butters and fat materials. Suitable wax materials for the compositions of the present invention are inert nontoxic materials with a melting point range between about 25 degrees C. and about 150 degrees C. and penetration at 25 degrees C. of about 1 to about 30. Examples of wax materials include natural waxes, synthetic waxes and mixtures thereof. Suitable waxes also include but are not limited to natural, regenerated, or synthetic food approved waxes including animal waxes such as beeswax, vegetable waxes such as carnauba, candelilla, sugar cane, rice bran, and bayberry wax, mineral waxes such as petroleum waxes including paraffin and microcrystalline wax, and mixtures thereof.

The lipids may or may not include at least one polyol fatty acid ester component. Examples of polyol fatty acid esters include but are not limited to triacylglycerides, propylene glycol diesters, and tetra esters of pentaerythritol. The lipids can include at least one polyol partial ester. Examples of suitable polyol partial esters include monoacylglycerides, diacylglycerides, and sorbitan partial esters (e.g., diesters and triesters of sorbitan). In some embodiments, the polyol fatty acid ester may include from 2 to 6 carbon atoms and 2 to 6 hydroxyl groups. Examples of suitable polyol fatty acid esters include glycerol, trimethylolpropane, ethylene glycol, propylene glycol, pentaerythritol, sorbitan and sorbitol. In certain embodiments, monoacylglycerides are compounds made up of a glycerol and a fatty acid bound as an ester. Diacylglycerols are compounds made up of a glycerol and two fatty acids; each fatty acid is bound to the glycerol as an ester. Triacylglycerides are compounds made up of a glycerol and three fatty acids, each fatty acid is bound to the glycerol as an ester. Fatty acids in the polyol esters of a natural oil include saturated fatty acids, as a non-limiting example, palmitic acid (hexadecanoic acid) and stearic acid (octadecanoic acid), and unsaturated fatty acids, as a non-limiting example, oleic acid (9-octadecenoic acid), linoleic acid (9,12-octadecadienoic acid), and linolenic acid (9,12,15-octadecatrienoic acid). Suitable fat materials and/or glyceride materials can also include phospholipids, sphingolipids, cholesterol and steroid derivatives, terpenes and vitamins.

The fat material of the present invention may include but is not limited to a glyceride selected from glyceryl monostearate, glyceryl tristearate and mixtures thereof. Other fat materials which can be used are hydrogenated palm oil, hydrogenated palm kernel oil, hydrogenated peanut oil, hydrogenated rapeseed oil, hydrogenated rice bran oil, hydrogenated soybean oil, hydrogenated cottonseed oil, hydrogenated sunflower oil, partially hydrogenated soybean oil, partially hydrogenated cottonseed oil, and mixtures thereof.

Examples of solid fat materials, which can be used in the present invention include but are not limited to solid hydrogenated castor and vegetable oils, hard fats, and mixtures thereof. Other fat materials, which can be used, include triglycerides of food grade purity, which can be produced by synthesis or by isolation from natural sources. Natural sources can include animal fat or vegetable oil, such as soy oil, as a source of long chain triglycerides (LCT). The solid fat materials can include butters, for example butters include Shea butter, cocoa butter and jojoba butter.

Other triglycerides suitable for use in the present invention are composed of a majority of medium length fatty acids (C10-C18), denoted medium chain triglycerides (MCT). The fatty acid moieties of such triglycerides can be unsaturated or polyunsaturated and mixtures of triglycerides having various fatty acid material. Phospholipids, which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and beta-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) can also be used.

Additional lipid compounds as fat material that can be used include but are not limited to tocopherol and derivatives, and oils and derivatized oils such as stearlyamine.

The fat material can be fatty acids and derivatives thereof which can include, but are not limited to, saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that can be used include, but are not limited to, molecules that have between 12 carbon atoms and 22 carbon atoms in either linear or branched form. Examples of saturated fatty acids that can be used include, but are not limited to, lauric, myristic, palmitic, and stearic acids. Examples of unsaturated fatty acids that can be used include, but are not limited to, lauric, physeteric, myristoleic, palmitoleic, petroselinic, and oleic acids. Examples of branched fatty acids that can be used include, but are not limited to, isolauric, isomyristic, isopalmitic, and isostearic acids and isoprenoids. Fatty acid derivatives include 12-(((7′-diethylaminocoumarin-3yl)carbonyl)methylamino)-octadecanoic acid; N-[12-(((7′diethylaminocoumarin-3-yl)carbonyl)methyl-amino)octadecanoyl]-2 -aminopalmitic acid, N succinyl-dioleoylphosphatidylethanol amine and palmitoyl-homocysteine; and/or combinations thereof. Mono, di and triglycerides or derivatives thereof that can be used include, but are not limited to, molecules that have fatty acids or mixtures of fatty acids between 6 and 24 carbon atoms, digalactosyldiglyceride, 1,2-dioleoyl-sn-glycerol; 1,2-cdipalmitoyl-sn-3 succinylglycerol; and 1,3-dipalmitoyl-2-succinylglycerol.

Examples of fatty alcohols that can be used include but are not limited to ethoxylated fatty alcohols, alkylphenols, ethoxylated alkylphenols, ethoxylated fatty amines, ethoxylated monoglycerides and ethoxylated diglycerides

The shell may comprise in part a polymer increasing bio-adhesion through electrostatic attraction and/or hydrophobic interactions to the surfaces of the skin or some combination thereof. The shell can comprise polymers including but not limited to any of the cationic conditioning agents known in the art.

Hydrocarbon cationic conditioner agents suitable for use herein are selected from the following classes of compounds:

(i) Cationic quaternary ammonium salts. The counterion is methyl sulfate or any alkyl sulfate or any halide. Examples of cationic quaternary ammonium salts include, but are not limited to:
(1) Acyclic quaternary ammonium salts having at least two C₈₋₃₀, preferably C₁₂₋₂₂ alkyl chains, such as: ditallowdimethyl ammonium methylsulfate, di(hydrogenated tallow)dimethyl ammonium methylsulfate, distearyldimethyl ammonium methylsulfate, dicocodimethyl ammonium methylsulfate and the like;
(2) Cyclic quaternary ammonium salts of the imidazolinium type such as di(hydrogenated tallow)dimethyl imidazolinium methylsulfate, 1-ethylene-bis(2-tallow-1-methyl) imidazolinium methylsulfate and the like;
(3) Diamido quaternary ammonium salts such as: methyl-bis(hydrogenated tallow amidoethyl)-2-hydroxyethyl ammonium methyl sulfate, methyl bis(tallowamidoethyl)-2-hydroxypropyl ammonium methylsulfate and the like;
(4) Biodegradable quaternary ammonium salts such as N,N-di (tallowoyl-oxy-ethyl)-N,N,-dimethyl ammonium methyl sulfate and N,N-di (tallowoyl-oxy-propyl)-N,N-dimethyl ammonium methyl sulfate.

The particularly preferred cationic conditioning agents for the spheres of the present invention are: behenyltrimethylammonium chloride; ditallowdimethylammonium methylsulfate; ditallowdimethylammonium chloride; methyl(1) stearylamidoethyl (2) stearylimidazolinium methosulfate; methyl(1) stearylamidoethyl(2)stearylimidazolinium chloride; N,N-di(tallowyl-oxy-ethyl)-N,N-dimethyl ammonium chloride; N,N-di(canolyl-oxy-ethyl)-N,N-dimethyl ammonium chloride; N,N-di(tallowyl-oxy-ethyl)-N-methyl, N-(2-hydroxyethyl) ammonium chloride; N,N-di(canolyl-oxy-ethyl)-N-methyl, N-(2-hydroxyethyl) ammonium chloride; N,N-di(2-tallowyloxy-2-oxo-ethyl)-N,N-dimethyl ammonium chloride; N,N-di(2-canolyloxy-2-oxo-ethyl)-N,N-dimethyl ammonium chloride; N,N-di(2-tallowyloxyethylcarbonyloxyethyl)-N,N-dimethyl ammonium chloride; N,N-di(2-canolyloxyethylcarbonyloxyethyl)-N,N-dimethyl ammonium chloride; N-(2-tallowoyloxy-2-ethyl)-N-(2-tallowyloxy-2-oxo-ethyl)-N,N-dimethyl ammonium chloride; N-(2-canolyloxy-2-ethyl)-N-(2-canolyloxy-2-oxo-ethyl)-N,N-dimethyl ammonium chloride; N,N,N-tri(tallowyl-oxy-ethyl)-N-methyl ammonium chloride; N,N,N-tricanolyl-oxy-ethyl)-N-methyl ammonium chloride; N-(2-tallowyloxy-2-oxoethyl)-N-(tallowyl)-N,N-dimethyl ammonium chloride; N-(2-canolyloxy-2-oxoethyl)-N-(canolyl)-N,N-dimethyl ammonium chloride; 1,2-ditallowyloxy-3-N,N,N-trimethylammoniopropane chloride; and 1,2-dicanolyloxy-3-N,N,N-trimethylammoniopropane chloride; and mixtures of thereof.

Methyl-1 -tallowamidoethyl-2-tallowimidazolinium methylsulfate, available from Witco Chemical Company under the name Varisoft™ 475. Examples of monoalkyltrimethylammonium salts are monotallowtrimethylammonium chloride, mono(hydrogenated tallow)trimethylammonium chloride, palmityltrimethyl ammonium chloride and soyatrimethylammonium chloride, available from Witco Chemical Company under the names Adogen™ 471, Adogen™ 441, Adogen™ 444, and Adogen™ 415, respectively. Examples of behenyltrimethylammonium chloride are commercially available under the name Kemamine™ Q2803-C from Humko Chemical Division of Witco Chemical Corporation. Methylbis(tallowamidoethyl)(2-hydroxyethyl)ammonium methylsulfate and methylbis(hydrogenated tallowamidoethyl)(2-hydroxyethyl)ammonium methylsulfate; are available from Witco Chemical Company under the names Varisoft™ 222 and Varisoft™ 110, respectively: dimethylstearylbenzyl ammonium chloride sold under the names Varisoft™ SDC by Witco Chemical Company and Ammonyx™ 490 by Onyx Chemical Company.

Preferred suitable materials for the shell include bio-adhesive polymers of polyquaternium 67, cocoamidopropyl dimethylamine, and hydroxyethyl behenamidopropyl dimonium chloride. The most preferred cationic surface-active agents for the shell are cetyl trimethylammonium chloride and behenamidopropyl hydroxyethyl dimonium chloride under the name Incroquat Behenyl HE®, commercially available from Croda Inc. The spheres of the present invention can comprise surfactants including esters of polyhydric alcohols such as sorbitan esters or glycerol stearate. Preferred sorbitan esters are monoalkyl. A common example of a sorbitan ester is SPAN 60 (ICI), which is a mixture of sorbitan and isosorbide stearates. Additional surfactants include mineral oils, and polyols such as polyethylene glycol, silicone oils and silicone surfactants.

Suitable surfactants or solubilizing excipient include but are not limited to non-volatile organic solvents, surfactants or combinations thereof which include but are not limited to sodium lauryl sulfate, cocoamidopropylbetaine, lauroamphoacetate, dialkylamine oxide, alkyl polyglycoside, methyl glucamide, sarcosinate, taurate, cocoyl isethionate, sucrose distearate, diglyceryldistearate, tetraglyceryl tristearate, decaglyceryl decastearate, diglyceryl monostearate, hexaglyceyl tristearate, decaglyceryl pentastearate, sorbitan monostearate, sorbitan tristearate, diethylene glycol monostearate, ester of glycerol and of palmitic acid, ester of glycerol and stearic acid, monostearate polyoxyethylenated containing 2 oxyethylene units, glyceryl mono- and dibehenate and pentaerythrityl tetrastearate, alkyl carboxylates, acyl lactylates, alkyl ether carboxylates, N-acyl sarcosinates, polyvalent alkyl carbonates, N-acyl glutamates, fatty acid, polypeptide condensates, sulfuric acid esters, polyoxyethylene, lecithin, ethoxylated alcohols, ethoxylated esters, ethoxylated amides, polyoxypropylene, propoxylated alcohol, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, sorbitan esters, sucrose esters, glucose esters and simethicone, aromatic esters and alkyl esters.

The surface-active agent can be a charged surfactant or non-ionic surfactant including anionic, cationic, nonionic, amphoteric, zwitterionic and combinations thereof. Examples of suitable amphoterics are cocoamidopropylbetaine and lauroamphoacetate. Examples of suitable nonionics are dialkylamine oxides, alkyl polyglycosides and methyl glucamides. Examples of anionic surfactants include salts of sarcosinate, taurate and cocoyl isethionate. Other surfactants that can be used in the delivery system of the present invention are sucrose distearate, diglyceryldistearate, tetraglyceryl tristearate, decaglyceryl decastearate, diglyceryl monostearate, hexaglyceyl tristearate, decaglyceryl pentastearate, sorbitan monostearate, sorbitan tristearate, diethylene glycol monostearate, the ester of glycerol and of palmitic acid and stearic acid, monostearate polyoxyethylenated containing 2 oxyethylene units, glyceryl mono- and dibehenate and pentaerythrityl tetrastearate.

Alternative anionic surfactants for use as surface active agents in the present invention include docusate salts such as the sodium salt thereof. Other suitable anionic surfactants include, without limitation, alkyl carboxylates, acyl lactylates, alkyl ether carboxylates, N-acyl sarcosinates, polyvalent alkyl carbonates, N-acyl glutamates, fatty acid, polypeptide condensates and sulfuric acid esters.

In other aspects of the invention amphoteric (amphipathic/amphiphilic surfactants), non-ionic surfactants and/or cationic surfactants can be used as the surface active agent in the coprocessed compositions of the present invention. Suitable pharmaceutically-acceptable non-ionic surfactants include, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., polysorbate esters), such as TWEEN 80, glucose (dextrose) esters and simethicone. The HLB for one acceptable non-ionic surfactant, polysorbate 40, is about 15.6.

A surfactant can be used which comprises between about 0.001% to about 20% by weight of the core. Preferably, the surfactant comprises about 1% to about 5% by weight of the core.

Other suitable pharmaceutically acceptable surfactants include acacia, benzalkonium chloride, cholesterol, emulsifying wax, glycerol monostearate, lanolin alcohols, lecithin, poloxamer, polyoxyethylene, and castor oil derivatives. Example emulsifiers include polyglyceryl-3 diisostearate.

### Active Ingredients

The active substances to be released by the composition can serve the dermal treatment of local skin diseases, the intradermal and transdermal treatment of diseases, the treatment of wounds, the treatment of conditions affecting the hair follicles, the treatment or the skin care in cosmetic preparations.

The spheres of the composition can include one or more cosmetic, dermatological, and pharmaceutical active ingredients that have an effect on the skin or hair follicles, including, but not limited to: anti-oxidants; free radical scavengers; moisturizers; depigmentation agents; reflectants; humectants; antimicrobial (e.g., antibacterial) agents; allergy inhibitors; anti-acne agents; anti-itch agents, anti-itch agents can be antihistamines such as diphenhydramine, steroids such as hydrocortisone, local anesthetics such as benzocaine or counterirritants such as menthol; anti-aging agents; skin brightening agents; anti-photoaging agents, anti-alopecia agents; anti-wrinkling agents, antiseptics; analgesics; antitussives; antipruritics; local anesthetics; anti-hair loss agents; hair growth promoting agents including minoxidil; hair growth inhibitor agents, antihistamines; keratolytic agents; anti-inflammatory agents; fresheners; healing agents; anti infectives; inflammation inhibitors; anticholinergics; vasoconstrictors; vasodilators; wound healing promoters; peptides, polypeptides and proteins; deodorants and antiperspirants; skin emollients and skin moisturizers; hair conditioners; hair softeners; hair moisturizers; tanning agents; skin lightening agents; antifungals such as antifungals for foot preparations; depilating agents; external analgesics; counterirritants; hemorrhoidals; insecticides; poison ivy products; poison oak products; burn products; anti-diaper rash agents; prickly heat agents; make-up preparations; vitamins; amino acids and their derivatives; herbal extracts; retinoids; flavoids; sensory markers (i.e., cooling agents, heating agents, etc.); skin conditioners; hair lighteners; chelating agents; cell turnover enhancers; coloring agents; sunscreens; anesthetics; immunomodulators and nourishing agents; moisture absorbers; sebum absorbers and the like, and mixtures thereof.

Local anaesthetics, local antibiotics, including for example clindamycin for treating folliculitis, antiseptics, antimycotics, antihistaminics, and antipruritic drugs; keratolytics and caustic drugs; virustatics, antiscabietic agents, steroids, as well as different substances for the treatment of acne, psoriasis, photodermatoses, or precancerous stages can be used with the composition of the present invention for the dermal treatment of local skin diseases. Active substances applicable by the intradermal route with the composition of the present invention include, for example, steroid and non-steroid antirheumatics, local anaesthetics, substances stimulating the blood flow, vasoprotectors and vasoconstrictors to treat vascular diseases, as well as active substances to influence processes in the subcutaneous fatty tissue. Transdermally applicable active substances to be used in the compostion of the present invention include, for example, analgesics, anti-arrhrythmic drugs, narcotics and their antagonists, neuroleptics, hormones or hormone substitutes, antidepressants, tranquilizers, hypnotics, psychostimulants, antiparkinson drugs, ganglionic blockers, sympathomimetics, alpha-sympatholytics, beta-sympatholytics, antisympathotonics, anti-asthmatics, antiemetics, appetite depressants, diuretics, or active substances for weight reduction, and the like. Because of the small thickness of the system according to the present invention preferred active substances are those developing their action already at very low concentrations. Examples of these preferred active substances include steroids, such as estradiol, estriol, progesterone, norethisterone, norethindrone, levonorgestrel and their derivatives, as well as estradiol diacetate, norgestamate, gestagens, desogestrel, demegestrone, promegestrone, testosterone, hydrocortisones and their derivatives; nitro compounds, such as amyl nitrate, nitroglycerin, isosorbide dinitrate; amine compounds, such as nicotine, chlorpheniramine, terfenadine, and triprolidine; oxicam derivatives such as piroxicam; mucopolysaccharases such as thiomucase; opioid substances such as buprenorphine, morphine, fentanyl and their salts, derivatives or analogues, naloxone, codeine, dihydroergotamine, lysergic acid derivatives, pizotiline, salbutamol, terbutaline; prostaglandins, such as PGA, PGB, PGE and the PGF-series, for example, misoprostol and enprostil, omeprazol, imipramine; benzamides, such as metoclopramines and scopolamine; peptides and growth factors such as EGF, TGF, PDGF, and the like; somatostatin; clonidin; dihydropyridines, such as nifedipine, nitrendipine, verapamil, diltiazem, ephedrine, propanolol, metoprolol, spironolactone; thiazides such as hydrochlorothiazide and flunarizine. Styptic active substances and wound-cleansing substances, such as enzymes, antiseptics, disinfectants, and antibiotics; pain-relieving agents and anaesthetic active substances, as well as active substances promoting wound healing to stimulate granulation, to induce vascularization, or to promote epithelization can be used with the composition of the present invention for the treatment of wounds.

The composition of the present invention can also comprise vegetable preparations, such as extracts or tinctures for the treatment of topical skin diseases. Suitable extracts or tinctures include oak bark extract, walnut extract, tincture of arnica, hamamelis extract, ribwort extract, pansy extract, thyme or sage extract; for the treatment of damaged or injured skin, for example, St. John's wort tincture, cone flowers tincture, chamomile flowers extract, or calendula flowers tincture; and for the care of exhausted and damaged skin, for example, birch leaves extract, nettle extract, coldsfoot extract, comfrey tincture, horsetail extract, or aloe vera extract. Vegetable preparations can also be released from the film layer for the intradermal treatment of diseases, for example, extracts of horse chestnut and butcher's broom in case of vein diseases, or extracts and tinctures of arnica, calendula, and capsicum in case of contusions, distortions, or haemorrhages. Vegetable preparations in the system according to the present invention may also be used in transdermal therapy, for example, ginseng extract in case of geriatric complaints; valerian tincture, extracts of melissa and hop to cause a sedative effect in case of superexcitation, sleep disturbances, and stress; extracts of kola and tea to achieve a stimulative effect; or hawthorn extract to stabilize the circulatory system.

Suitable amino acid agents that can be used with the spheres of the present invention include amino acids derived from the hydrolysis of various proteins as well as the salts, esters, and acyl derivatives thereof. Examples of such amino acid agents include amphoteric amino acids such as alkylamido alkylamines, stearyl acetyl glutamate, capryloyl silk amino acid, caprylol collagen amino acids; capryloyl kertain amino acids; capryloyl pea amino acids; cocodimonium hydroxypropyl silk amino acids; corn gluten amino acids; cysteine; glutamic acid; glycine; hair keratin amino acids; hair amino acids such as aspartic acid, threonine, serine, glutamic acid, proline, glycine, alanine, half-cystine, valine, methionine, isoleucine, leucine, tyrosine, phenylalanine, cysteic acid, lysine, histidine, arginine, cysteine, tryptophan, citrulline; lysine; silk amino acids, wheat amino acids; and mixtures thereof.

Suitable peptides, polypeptides, and proteins that can be used with the spheres of the present invention include small oligopeptides as well as those polymers that have a long chain, such as at least about 10 carbon atoms, and a high molecular weight, such as at least about 500 Da. Examples of such peptides and proteins include palmitoyl tripeptide-5, collagen, deoxyribonuclease, iodized corn protein; keratin; milk protein; protease; serum protein; silk; sweet almond protein; wheat germ protein; wheat protein; wheat protein, alpha and beta helix of keratin proteins; hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Examples of suitable vitamins that can be used with the spheres of the present invention include but are not limited to vitamin B complex; including thiamine, nicotinic acid, biotin, pantothenic acid, choline, riboflavin, vitamin B6, vitamin B12, pyridoxine, inositol, carnitine; vitamins A, C, D, E, K and their derivatives such as vitamin A palmitate and pro-vitamins, such as panthenol (pro vitamin B5) and panthenol triacetate, and mixtures thereof.

Examples of suitable antibacterial agents that can be used with the spheres of the present invention include but are not limited to bacitracin, erythromycin, neomycin, tetracycline, chlortetracycline, benzethonium chloride, phenol, and mixtures thereof.

Examples of suitable skin emollients and skin moisturizers that can be used with the spheres of the present invention include but are not limited to mineral oil, lanolin, vegetable oils, isostearyl isostearate, glyceryl laurate, methyl gluceth 10, methyl gluceth 20 chitosan, and mixtures thereof.

Examples of suitable external analgesics and local anesthetics that can be used with the spheres of the present invention include but are not limited to benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof.

Examples of suitable antiperspirants and deodorants that can be used with the spheres of the present invention include but are not limited to aluminium chlorohydrates, aluminium zirconium chlorohydrates, and mixtures thereof.

Examples of suitable counterirritants that can be used with the spheres of the present invention include but are not limited to camphor, menthol, methyl salicylate, peppermint and clove oils, ichtammol, and mixtures thereof.

Examples of suitable hemorrhoidal products that can be used with the spheres of the present invention include but are not limited to anesthetics such as benzocaine, pramoxine hydrochloride, and mixtures thereof; antiseptics such as benzethonium chloride; astringents such as zinc oxide, bismuth subgallate, balsam Peru, and mixtures thereof; skin protectants such as cod liver oil, vegetable oil, and mixtures thereof.

A type of benefit agent that can be used with the spheres of the present invention includes but is not limited to those therapeutic agents that are effective in the treatment of dandruff, seborrheic dermatitis, and psoriasis as well as the symptoms associated therewith. Examples of such suitable therapeutic agents include but are not limited to zinc pyrithione, shale oil and derivatives thereof such as sulfonated shale oil, selenium sulfide, sulfur; salicylic acid; coal tar; povidone-iodine and imidazoles.

Antimicrobials that can be used with the spheres of the present invention for topical application include but are not limited to penicillins, cephalosporins, other beta-lactam compounds, aminoglycosides, tetracyclines, erythromycin, antifungal agents, and the like and a combination thereof.

Antiseptics that can be used with the spheres of the present invention for topical application onto acneiform skin include but are not limited to triclosan (Irgasan DP 300), phenoxy isopropanol, resorcinol, chlorhexidine, povidone iodine.

Keratolytic agents that can be used with the spheres of the present invention for topical application onto acneiform skin include but are not limited to salicylic acid, benzoyl peroxide, sulphur, retinoic acid and any of a number of fruit acids and alpha hydoxy acids.

Anti-irritants that can be used with the spheres of the present invention for the topical application onto acneiform skin include but are not limited to alpha-bisabolol, farnesol, chamomile extract and glycyrrhetinic acid.

Examples of anti-inflammatory analgesic agents that can be used with the spheres of the present invention include but are not limited to acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexarnac, piroxicam, phenylbutazone, oxyphenbutazone, clofezone, pentazocine, mepirizole, tiaramide hydrochloride, and the like. Examples of steroidal anti-inflammatory agents that can be used with the spheres of the present invention include but are not limited to hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, and the like.

Examples of antihistamines that can be used with the spheres of the present invention include but are not limited to diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, and the like. Examples of local anesthetics that can be used with the spheres of the present invention include but are not limited to dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, and the like.

Examples of bactericides and disinfectants that can be used with the spheres of the present invention include but are not limited to thimerosal, phenol, thymol, benzalkonium chloride, benzethonium chloride, chlorhexidine, povidone iodine, cetylpyridinium chloride, eugenol, trimethylammonium bromide, and the like. Examples of vasoconstrictors that can be used with the spheres of the present invention include but are not limited to naphazoline nitrate, tetrahydrozoline hydrochloride, oxymetazoline hydrochloride, phenylephrine hydrochloride, tramazoline hydrochloride, and the like. Examples of hemostatics that can be used with the spheres of the present invention include but are not limited to thrombin, phytonadione, protamine sulfate, aminocaproic acid, tranexamic acid, carbazochrome, carbaxochrome sodium sulfanate, rutin, hesperidin, and the like.

Examples of chemotherapeutic drugs that can be used with the spheres of the present invention include but are not limited to sulfamine, sulfathiazole, sulfadiazine, homosulfamine, sulfisoxazole, sulfisomidine, sulfamethizole, nitrofurazone, and the like. Examples of antibiotics that can be used with the spheres of the present invention include but are not limited to penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, and the like.

Examples of antiviral drugs that can be used with the spheres of the present invention include but are not limited to protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Example of cosmetic active ingredients that can be used with the spheres of the present invention include but are not limited to D-alpha-tocopherol, DL-alpha-tocopherol, D-alpha-tocopheryl acetate, DL-alpha-tocopheryl acetate, ascorbyl palmitate, vitamin F and vitamin F glycerides, vitamin D, vitamin D₂, vitamin D₃, retinol, retinol esters, retinyl palmitate, retinyl propionate, beta-carotene, D-panthenol, famesol, farnesyl acetate; jojoba oils and blackcurrant oils rich in essential fatty acids; 5-n-octanoylsalicylic acid and esters thereof, salicylic acid and esters thereof; alkyl esters of .alpha.-hydroxy acids such as citric acid, lactic acid, glycolic acid; asiatic acid, madecassic acid, asiaticoside, total extract of Centella asiatica, beta-glycyrrhetinic acid, alpha-bisabolol, ceramides such as 2-oleoylamino-1,3-octadecane; phytanetriol, phospholipids of marine origin which are rich in polyunsaturated essential fatty acids, ethoxyquine; extract of rosemary, extract of balm, quercetin, extract of dried microalgae, anti-inflammatory agents, such as steroidal anti-inflammatory agents, and biostimulants, for example hormones or compounds for the synthesis of lipids and/or proteins.

Skin treating compositions can be used in the spheres of the present invention. Skin treating compositions can include but are not limited to vitamin C, vitamin E, and optionally, alpha-hydroxy acids, such as lactic and glycolic acids and other keratinolytics for the treatment or prevention of wrinkles and skin dryness.

Vitamin C has many uses in skin care, including but not limited to promoting collagen (connective tissue) synthesis, lipid (fat) and carbohydrate metabolism, and the synthesis of neurotransmitters. It is also essential for optimum maintenance of the immune system. Vitamin C is toxic to a wide range of cancer cells, especially melanoma. The oxidizing enzyme tyrosinase that catalyzes the aerobic conversion of tyrosine into melanin and other pigments is also inhibited by the presence of vitamin C. Besides the many applicable uses set forth above, vitamin C is essential for collagen synthesis and wound healing. The spheres of the present invention can comprise a combination of vitamin C, vitamin E and other ingredients, such as moisturizers, collagen synthesis promoting agents and exfoliating agents.

Skin conditioners, moisturizers and surfactants can be included as additives in the spheres of the present invention. Illustrative conditioners include but are not limited to mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

The concentration of the active ingredient in the drug delivery system of the present invention depends on the desired treatment strength. Typically, this concentration can range from about 0.001% to about 80% by weight relative to the total weight of the sphere. Preferably, this percentage is in the range of about 1% to about 50%.

Plasticizers, penetration enhancers, as described in the text “Transdermal Delivery of Drugs, A. F. Kydonieus (ED) 1987 CRL Press and in U.S. Pat. Nos. 4,913,905, 4,917,676 and 5,032,403 hereby incorporated by reference into this application, coloring agents, and preservatives can be included in the delivery system of the present invention and comprise no more than about 10% of the final weight of the spheres, but the amount can vary depending on the active ingredient or other components. Glycerin, which is also a moisturizing agent, can be added as an anti-irritant or to modulate the delivery of the other skin treating agents and can be present in amounts of from about 0 to about 20% by weight.

Examples of encapsulated active ingredients in water sensitive micro-spheres are spray dried active ingredients with starch and other natural or synthetic water-soluble or water-dispersible polymers. On contact with skin moisture, the spray dried micro-spheres, comprising the active ingredients, are released, thereby promoting the controlled delivery or the enhanced bioavailability of active ingredients. Examples of encapsulated ingredients in spheres are dispersions of hydrophobic materials, as described above, such as lipids, waxes, and hydrophobic polymers comprising active ingredients in the hydrophobic matrix. On contact with skin moisture, the hydrophobic spheres containing the active ingredients are released, thereby allowing the controlled and sustained delivery or the enhanced bioavailability of active ingredients over time.

The composition of the present invention can be used for the treatment of follicular skin diseases and infection. In one embodiment, the composition of the present invention can be used for the treatment of acne. In one embodiment, the composition of the present invention can be used for the treatment of folliculitis. In one embodiment, the composition of the present invention can be used for the treatment of rosacea. In one embodiment, the composition of the present invention can be used for the treatment of hidradentitis suppurativa. In one embodiment, the composition of the present invention can be used for the treatment of Keratosis pilaris. In one embodiment, the composition of the present invention can be used for the treatment all forms of alopecia including but not limited to: androgenic alopecia, cicatricial alopecia, alopecia areata, and chemotherapy-induced alopecia. In one embodiment, the composition of the present invention can be used for the treatment of itching. In one embodiment, the composition of the present invention can be used for the local treatment of pain using painkillers including but not limited to non-steroidal anti-inflammatory compounds. In one embodiment, the composition of the present invention can be used for the treatment of dandruff. In one embodiment, the composition of the present invention can be used for administering local anesthetic.

The dermatological drug delivery system of the present invention can be used for topical use in a formulation free of volatile organic solvents like ethanol, methanol.

The delivery system of the present invention allows encapsulation of both hydrophobic and hydrophilic actives.

This invention is designed for a number of embodiments in the field of dermatological treatment within prescription pharmaceutical, over-the-counter pharmaceutical and consumer materials. The composition of the present invention comprises microspheres of diameters lower than about 10 μm which may be delivered in a variety of topical formulas, including but not limited to creams, lotions, ointments, shampoos, conditioners, and washes.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

### Minoxidil Penetration from Delivery System of the Present Invention Compared to Conventional Benchmark Product with Free Minoxidil

A delivery system of the present invention for the treatment of various alopecia conditions had the following composition: encapsulation of minoxidil in a matrix of lipid material and waxy emulsifiers with a cationic polymer shell. The specific composition is shown in table 1. Such an encapsulation enables the suspension of the active material in water-based, alcohol-free formulations. The resultant capsule, with a median particle size below 1μm, is well suited in size and composition to deliver the minoxidil deep within the hair follicle. The main advantage of this embodiment is the protection of the scalp, not from the active material, but from the solvents used as solubilizers and penetration enhancers as used in, for example, the conventional product Men's Rogaine® (trademark belongs to McNeill-PPC).

TABLE 1
Composition of the present invention (“encapsulated
minoxidil”) used in examples 1, 2, 3 and 4.

| Ingredient | Weight % |
|---|---|
| Minoxidil | 10 |
| Lactic Acid 88% in water | 35 |
| Water | 6 |
| Cocoamidopropyl dimethylamine | 35 |
| PEG 100 Stearate | 3.1 |
| Glyceryl monostearate | 1.9 |
| Beeswax | 8 |
| Preservative | 1 |

TABLE 2
The composition of leave-on conditioner used for the
penetration studies for the encapsulated minoxidil.

| Ingredient | Weight % |
|---|---|
| Water | 87.05 |
| Citric acid | 0.1 |
| Glycerin | 2 |
| Guar gum | 0.75 |
| Capric caprylic triglycerides | 2 |
| Sunflower seed oil | 0.1 |
| Jojoba oil | 1 |
| Cetyl alcohol | 1 |
| Stearyl alcohol | 5 |
| Preservative | 1 |

TABLE 3
The composition of encapsulated minoxidil in conditioner
used in the penetration studies in examples 1, 3 and 4.

| Ingredient | Weight % |
|---|---|
| Composition in table 1 | 50 |
| (leave-on conditioner) | |
| Composition in table 2 | 50 |
| (encapsulated minoxidil) | |

Penetration testing of the composition of the present invention and the Men's Rogaine® product using ex-vivo porcine skin as the membrane in a Franz diffusion cell demonstrated that the composition of the present invention provides skin penetration statistically indistinguishable from that of Men's Rogaine®. The penetration of minoxidil via the composition of the present invention is accomplished however without the use of ethanol (which is used in the Rogaine formula) to solubilize the minoxidil and disrupt the membranes of the skin.

### Example 2

### Minoxidil Penetration into Hair Follicles Visualized Using Confocal Fluorescent Microscopy

Ex-vivo skin testing was used to determine follicular penetration. A fluorescent dye, LD688, was encapsulated in the composition of the present invention described in Example 1, which was then examined for follicular penetration using con-focal fluorescent microscopy. The fluorescent marked capsules were applied to skin tissue, which was assessed at focal planes of increasing depth by con-focal fluorescent microscopy. These images establish qualitatively the penetration of the spheres into the follicle at depths equal to the detection limit of the microscopy technique.

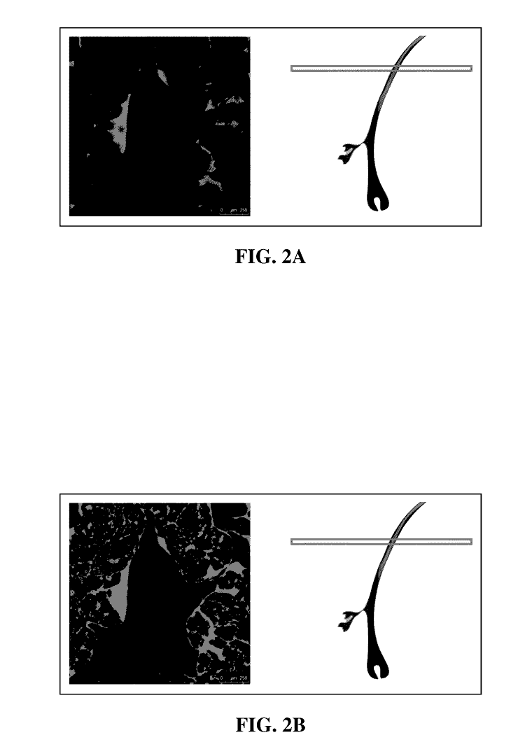

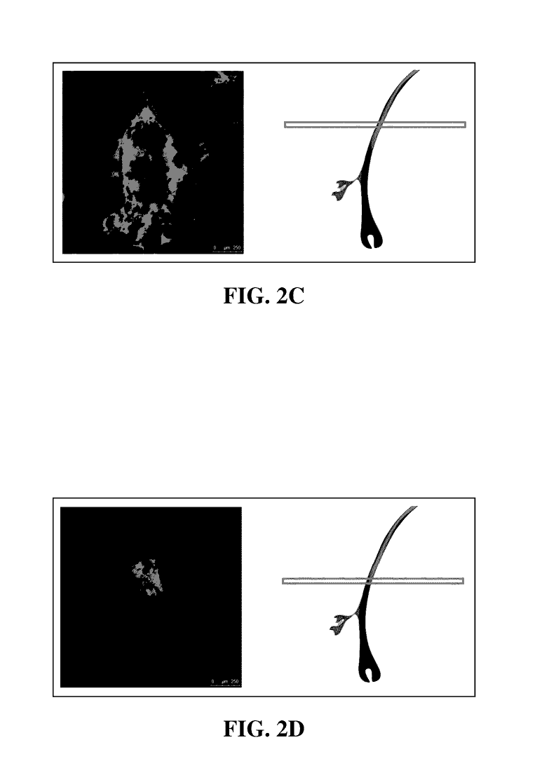

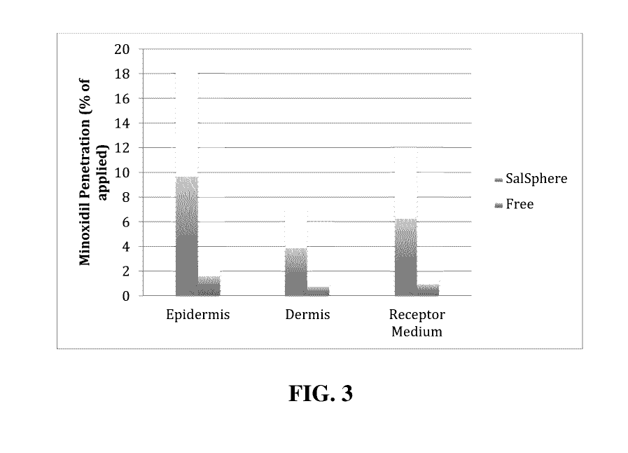

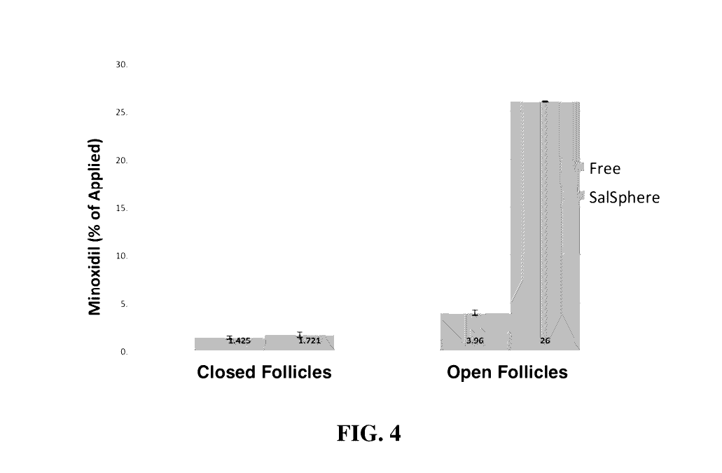

FIGS. 2A-2D show the two routes of penetration. After 24 hours very little material remains at the surface of the skin (FIG. 2A). Material placed around the follicle demonstrates slow penetration through the intercellular space of the stratum corneum, seen in FIG. 2B. However, FIG. 2C and FIG. 2D demonstrate the greater penetration achieved by passing along the follicle. Furthermore, FIG. 2D establishes the capacity of the composition of the present invention to deliver material down to the stratum basale, which is the detection limit of the con-focal microscopy technique due to light scattering.

### Example 3

### Minoxidil Penetration into the Skin Layers and Transdermally

To provide quantitative data of minoxidil penetration, and to ascertain if the composition of the present invention penetrates through the epidermis and into the dermis, a cross sectional penetration assay was performed. This experiment utilized ex-vivo porcine skin as the membrane of a Franz diffusion cell. Skin was treated with a composition of the present invention described in Example 1 (Table 3) and non-encapsulated API (Table 4), under the experimental conditions for 6 hours.

TABLE 4
The composition of reference formulation of minoxidil used
in the penetration studies (as “free minoxidil”).

| Ingredient | Weight % |
|---|---|
| Water | 71 |
| Cocoamidopropyl dimethylamine | 10 |
| Lactic acid | 10 |
| Amodimethicone | 2 |
| Cetyl trimethyl ammonium chloride | 2 |
| Minoxidil | 5 |

Thereafter, the skin samples were separated such that the dermis and epidermis could be assayed separately for minoxidil content. The results indicated that the composition of the present invention are shown in Table 1. The composition of the present invention provided an almost fivefold increase in penetration to the dermis, and a sevenfold increase in penetration to the epidermis, as shown in Table 5. Without encapsulation or penetration enhancers only 4.0% of the minoxidil applied penetrates into the skin. Using a composition of the present invention in the same test, 26.0% of the minoxidil penetrated into the skin. Of this 26%, 7.4% reached the dermal layer. More minoxidil penetrated into the dermis via the encapsulation of the composition of the present invention than penetrated both layers of the skin without encapsulation. Thus, a significant level of encapsulated material passes beyond the detection limit of fluorescent microscopy and into the dermis and into the receptor chamber, meaning transdermal penetration.

TABLE 5
Penetration enhancement afforded by this embodiment
of the technology, by layer of the skin.

| | Penetration from | Penetration from | Fold enhancement |
|---|---|---|---|
| | encapsulated | free | of penetration by |
| | minoxidil (as % | minoxidil (as % | the present |
| | of applied) | of applied) | invention over |
| Skin layer | after 6 hours | after 6 hours | free minoxidil |
| Epidermis | 18.6 | 2.7 | 7 |
| (top layer) | | | |
| Dermis | 7.4 | 1.3 | 6 |
| (bottom layer) | | | |
| Through skin | 12.0 | 1.6 | 8 |
| into receptor | | | |
| medium | | | |

### Example 4

### Minoxidil Penetration by Transfollicular Delivery

To confirm that the skin penetration observed in the quantitative testing follows the same mechanism as the penetration shown by fluorescent microscopy, a negative control for follicular penetration was established. This was accomplished by sealing the follicles of ex-vivo porcine skin with a polymer-based lacquer before applying the composition of the present invention, described in Example 1, and assessing any change in penetration.

The results of this assay showed a decrease from 26-35% of applied material penetrating into the skin with encapsulation to less than 2% of the applied minoxidil penetrating into the skin, as shown in Table 6. This validates the hypothesis that closing the follicles greatly decreases the penetration, confirming the hypothesis that the increased penetration seen is due to follicular delivery.

TABLE 6
Penetration of minoxidil through open and closed follicles into the
skin from the present invention as compared to free minoxidil.

| | Penetration from | Penetration from | Fold enhancement |
|---|---|---|---|
| | encapsulated | free | of penetration by |
| | minoxidil (as % | minoxidil (as % | the present |
| | of applied) | of applied) | invention over |
| Condition | after 6 hours | after 6 hours | free minoxidil |
| Open follicles | 26 | 4 | 7 |
| Closed follicles | 1.8 | 1.4 | 1.3 |

This line of testing provides support for the benefits claimed by this technology: Non-irritating compositions without harsh solvents, providing equivalent or superior drug efficacy by means of follicular penetration to the deepest levels of the epidermis and into the dermis.

### Example 5

### A Topical Formulation to Deliver Minoxidil

A topical formulation to deliver minoxidil, an FDA-approved hair regrowth active, to the hair follicles to promote hair growth or regrowth is disclosed in Table 1.

### Example 6

### A Topical Formulation to Fight Keratosis Pilaris

Keratosis pilaris is a disorder marked by excess production of keratin that then seals up the hair follicles, usually resulting in red bumps. A formulation for a keratolytic treatment is shown in Table 7. Glycolic acid is combined with a topical retinoid such as tretinoin, retinyl palmitate. The keratolytic removes the blockage of the follicles, and allows the penetration of the retinoid.

TABLE 7
Topical formulation containing a keratolytic
and a retinoid to fight keratosis pilaris.

| Ingredient | Wt % |
|---|---|
| Water | 84.9 |
| Tretinoin | 1 |
| Polyglyceryl-3 diisostearate | 2 |
| Shea butter | 4 |
| Glyceryl monostearate | 6 |
| Polysorbate 80 | 2 |
| Softcat polymer SK-MH | 0.1 |
| total | 100 |

### Example 7

### A Topical Formulation to Fight Acne

Benzoyl peroxide is typically used to fight acne, it kills P. acnes bacteria that reside in the hair follicles. A formulation for acne treatment is shown in Table 8.

TABLE 8
Topical formulation containing benzoyl peroxide to fight acne.

| Ingredient | Wt % |
|---|---|
| Water | 85 |
| Benzoyl peroxide | 5 |
| Beeswax | 8 |
| Glyceryl monostearate | 4 |
| Incroquat Behenyl HE | 4 |
| Polysorbate 80 | 2 |
| total | 100 |

### Example 8

### A Topical Formulation to Fight Folliculitis

Folliculitis is a group of diseases characterized by an infection of the hair follicles. Typical treatments include antibiotics. A topical formulation of the present invention containing an antibiotic used to treat folliculitis, clindamycin, is shown in Table 9.

TABLE 9
Topical formulation containing clindamycin to fight folliculitis.

| Ingredient | Wt % |
|---|---|
| Water | 79.8 |
| Clindamycin | 10 |
| Glyceryl monostearate | 4 |
| PEG100 stearate | 4 |
| Polysorbate 80 | 2 |
| Softcat polymer SK-MH | 0.2 |
| total | 100 |

### Example 9

### A Topical Formulation to Fight Rosacea

Rosacea is a disease of the follicles that is characterized by redness/inflammation of the skin and can be treated with topical retinoids. A topical formulation for treating rosacea is shown in Table 10.

TABLE 10
Topical formulation containing a retinoid to fight rosacea.

| Ingredient | Wt % |
|---|---|
| Water | 84.9 |
| Tretinoin | 1 |
| Polyglyceryl-3 diisostrearate | 2 |
| Shea butter | 4 |
| Glyceryl monostearate | 6 |
| Polysorbate 80 | 2 |
| Softcat polymer SK-MH | 0.1 |
| total | 100 |

### Example 10

### A Topical Formulation to Fight Hidradentitis Suppurative

Hidradentitis suppurativa is a disease where the follicles become blocked; the causes are unclear but probably have a genetic component. Hidradentitis suppurativa sometimes treated with TNF-alpha inhibitors or injected steroids; these treatments are inconvenient and can have side effects, especially the TNF-alpha inhibitors, which are immunosuppressants. A preferred composition including topical antibiotics for treatment of hidradentitis suppurative is shown in Table 11. In this formula, lactic acid is added to function as a moisturizing and exfoliating agent, hydrating the skin and helping to unblock the follicles to allow the antibiotic to penetrate.

TABLE 11
Topical formulation containing clindamycin and lactic acid.

| Ingredient | Wt % |
|---|---|
| Water | 77.8 |
| Clindamycin | 10 |
| Lactic acid | 2 |
| Glyceryl monostearate | 4 |
| PEG100 stearate | 4 |
| Polysorbate 80 | 2 |
| Softcat polymer SK-MH | 0.2 |
| Total | 100 |

### Example 11

### A Topical Formulation to Fight Dandruff

Dandruff is caused by a fungal infection and can be treated by antifungals such as suprapein and salicylic acid. A topical formulation for treating dandruff is shown Table 12.

TABLE 12
Topical formulation containing suprapein
and salicylic acid to fight dandruff.

| Ingredient | Wt % |
|---|---|
| Water | 53.55 |
| SLES | 30 |
| Xanamide | 3 |
| Suprapein | 0.45 |
| Salicylic acid | 3 |

### Example 12

### A Topical Formulation to Fight Itching

A topical formulation of the present invention for treating itching is shown in Table 13.

TABLE 13
Topical formulation containing diphenhydramine
to combat itching.

| Ingredient | Wt % |
|---|---|
| Water | 85.9 |
| Diphenhydramine | 2 |
| Beeswax | 8 |
| Glyceryl monostearate | 4 |
| Softcat polymer SK-MH | 0.1 |
| total | 100 |

### Example 13

### A Topical Formulation for the Local Treatment of Pain Using Non-Steroidal Anti-Inflammatories

Non-steroidal anti-inflammatories such methyl salicylate can be applied topically to kill pain locally. A topical formulation of the present invention for treating pain is shown in Table 14.

TABLE 14
Topical formulation containing methyl
salicylate to kill pain topically.

| Ingredient | Wt % |
|---|---|
| Water | 87.9 |
| Methyl salicylate | 2 |
| Beeswax | 4 |
| Shea butter | 2 |
| Glyceryl monostearate | 4 |
| Softcat polymer SK-MH | 0.1 |
| total | 100 |

### Example 14

### A Topical Formulation for Administering Local Anesthetic

Local anesthetics that can be delivered by the present invention include but are not limited to benzocaine and lidocaine. A topical formulation of the present invention to administer a local anesthetic is shown in Table 15.

TABLE 15
Topical formulation containing benzocaine
to administer local anesthetic.

| Ingredient | Wt % |
|---|---|
| Water | 87.4 |
| Benzocaine | 10 |
| Beeswax | 8 |
| Shea butter | 6 |
| Glyceryl Monostearate | 4 |
| Polysorbate 80 | 0.5 |
| Softcat polymer SK-MH | 0.1 |
| total | 100 |

### INDUSTRIAL APPLICABILITY

The present invention comprises topical formulations that can treat diseases of the hair follicles including but not limited to acne, rosacea, folliculitis, hidradentitis suppurative, keratosis pilaris, and various alopecias. The invention offers targeted delivery to the follicles, which reduces irritation of healthy skin areas and allows the use of minimal levels of active ingredients for maximum effect.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A drug delivery system for use in a topical application to skin or hair follicles comprising a composition of spheres having a solid core and a semi-liquid shell surrounding the core, the spheres having a particle size of about 0.01 to about 10 micron, the solid core containing an active agent.

2. The drug delivery system of claim 1 wherein the core is hydrophobic, comprising one or more waxes, butters, hydrophobic polymers, hydrophobic co-polymers or mixtures thereof.

3. The drug delivery system of claim 1 wherein the shell coats the core via physical interactions, the shell comprises water-suspendable polymers.

4. The drug delivery system of claim 3 wherein the shell has bioadhesive properties through electrostatic attraction and hydrophobic interactions to the surfaces of the skin or the hair follicle.

5. The drug delivery system of claim 1 wherein a payload of the active agent is about 0.001% to about 80%.

6. The drug delivery system of claim 1 wherein the composition is a water-based formulation.

7. The drug delivery system of claim 1 wherein the composition is a water-based emulsion or suspension.

8. The drug delivery system of claim 1 wherein the spheres comprise:
between about 0.1% and 90% by weight of one or more lipids; between about 0.01% and 80% by weight bio-adhesive polymer;
between about 0.001% and 20% by weight of one or more surfactants;
between about 0.001% and 80% by weight of one or more of the active agents;
optionally a solubilizing excipient in an amount by weight of 0.01% to 90.

9. The drug delivery system of claim 8 wherein the solubilizing excipient is chosen from the group of glycerol esters, alkyl esters, and aromatic esters.

10. The drug delivery system of claim 8 where the lipid is chosen from the group of waxes, butters and fats.

11. The drug delivery system of claim 8 wherein the lipid is selected from the group consisting of fatty acid esters fatty acid esters, fatty alcohols, natural waxes, animal waxes, synthetic waxes, triglycerides, hydrogenated plant oils, and biodegradable natural polymers.

12. The drug delivery system of claim 8 where the surfactant is chosen from the group of consisting of sorbitan esters, mineral oils and polyols.

13. The drug delivery system of claim 8 where the suitable surfactants or solubilizing excipient are not volatile organic solvents.

14. The drug delivery system of claim 8 where the bio-adhesive polymer is chosen from the group of consisting of polyquaternium 67, cocoamidopropyl dimethylamine, and hydroxyethyl behenamidopropyl dimonium chloride.

15. The drug delivery system of claim 1 where the active agent is chosen from the group consisting of anti-acne agents, anti-aging agents, antibacterial agents, anesthetic agents, analgesic agents, anti-fungal agents, anti-photoaging agents, skin brightening agents, anti-itch agents, and anti-inflammatory agents.

16. The drug delivery system of claim 1 wherein the spheres are additionally encapsulated in a protective outer microsphere shell.

17. The drug delivery system of claim 16 where the shell is comprised of a moisture-sensitive material.

18. The drug delivery system of claim 1 that enables the delivery of the drug into the hair follicles at higher concentration than without the delivery system.

19. A skin or hair care product comprising the drug delivery system of claim 1.

20. A method for the treatment of a skin or hair condition comprising:
applying the drug delivery system of claim 1 to the skin or hair follicles of a subject to topically delivery a therapeutically effective amount of the active agent.

21. The method of claim 20 wherein the skin condition is acne.

22. The method of claim 20 wherein the skin condition is rosacea.

23. The method of claim 20 wherein the skin condition is hidradentitis suppurativa.

24. The method of claim 20 wherein the skin condition is keratosis pilaris.

25. The method of claim 20 wherein the skin condition is alopecia.

26. The method of claim 25 wherein the alopecia is selected from the group consisting of androgenic alopecia, cicatricial alopecia, alopecia areata, and chemotherapy-induced alopecia.

27. The method of claim 20 wherein the skin condition is itching.

28. The method of claim 20 wherein the skin condition is folliculitis.

29. The method of claim 20 wherein the skin condition is dandruff and/or seborrheic dermatitis.

30. The method of claim 20 wherein the applied delivery system is effective for local treatment of pain.

31. The method of claim 30 wherein the active agent is a non-steroidal anti-inflammatory compounds.

32. The method of claim 20 wherein the applied delivery system is effective for administering local anesthetic.
